(19) United States
(12) Patent Application Publication
(10) Pub. No.: US 2011/0170061 A1
(43) Pub. Date: Jul. 14, 2011

(54) Gaze Point Tracking Using Polarized Light

(76) Inventor: Gary B. Gordon, Saratoga, CA (US)

(21) Appl. No.: 12/788,058

(22) Filed: May 26, 2010

Related U.S. Application Data
(63) Continuation-in-part of application No. 12/684,613, filed on Jan. 8, 2010.

(51) Int. Cl. A61B 3/14

(52) U.S. Cl. 351/206

(57) ABSTRACT

A process and a system that determine a glint position and a pupil position are disclosed. The process includes illuminating one eye to produce a glint on that eye, and obtaining a glint image of that eye showing that glint on that eye. A glint position is determined at least in part from that glint image. The process further includes illuminating that eye using polarized light, and obtaining, through a polarizer that can attenuate reflected polarized light, a pupil image of that eye. A pupil position is determined at least in part from that pupil image.

### RELATED APPLICATION

The present application is a continuation-in-part of U.S. application Ser. No. 12/684,613, filed on Jan. 8, 2010, pending, titled “Gaze Tracking Using Polarized Light”, which is incorporated herein by reference in its entirety, and to which application we claim priority under 35 USC §120.

### BACKGROUND

A number of techniques for determining a viewer's gaze point by using eye tracking have been disclosed in the prior art. Some of them allow a person to control aspects of their environment by using eye movement. For example, a quadriplegic might use such a system for controlling his or her computer or other device to facilitate reading, communicating, and performing other useful tasks.

One class of gaze-tracking techniques uses artificial illumination to produce a glint reflection in the cornea of an eye. A camera captures an image of the eye, and in the image the relative positions of the centers of the pupil and glint are measured, as being indicative of the user's gaze direction or gaze point.

In the prior art, the glint and the pupil are captured in the same exposure. (Hereafter, an image of the pupil will generally be meant to mean an image including the iris). The exposure of the pupil and glint must be a compromise, because their brightness vary considerably, with the glint being by far the brightest. To reveal the periphery of the pupil, the iris requires a relatively substantial exposure, especially if the iris is dark. Unfortunately this can overexpose the glint and cause lens flare, which scatters and reflects light within the lens and result in blurring and other artifacts in the image. In the prior art, the exposure has been a compromise unlikely to produce an optimum image of either the pupil or the glint. The accuracy of such a system can suffer accordingly, and if used to control a computer screen, results in less precise control of the cursor.

Such systems, to minimize the problems caused by the glint artifacts, generally localize the pupil by primarily searching its upper periphery. This technique, however, breaks down if the user has the not uncommon condition known as drooping eyelid.

### SUMMARY OF THE INVENTION

In the present invention, the pupil and glint are imaged in a manner that allows their exposures to be independently optimized. Advantage is taken of two facts, first the fact that the glint is revealed by reflected light, whereas the pupil and iris are revealed by scattered light, and second, the fact that reflected light preserves polarization whereas scattered light does not. Since the glint is brighter than the iris, and is revealed by polarized light, it can be selectively attenuated as much as desired, and overexposure prevented. Accordingly, higher quality images of the pupil and glint can be captured, allowing for a more accurate determination of their center and centroids, and consequentially a more stable and accurate determination of the user's gaze target.

In the present invention, a pupil illuminator is fitted with a polarizer, and used to flood the user's face with polarized light, which may be infrared light. An image or images of the eye or eyes is then captured by a camera that is also fitted with a polarizer. Light from this pupil illuminator is scattered from the pupil and iris, and is captured in a pupil image. The exposure is adjusted so as to reveal the detail of the pupil and iris, and in particular to expose the iris at middle gray values such that it is clearly delineated against the pupil. Even though the light from the pupil illuminator is polarized, the scattering from the pupil and iris depolarize it, such that the camera will capture the pupil and iris in the pupil image regardless of the orientation of the camera's polarizer. Light from the pupil illuminator will also produce a bright glint on the cornea. Since this glint is a consequence of a reflection, it is polarized. It is the purpose of the polarizer on the camera to reduce or obliterate all traces of this glint in the pupil image, which is accomplished by adjusting its orientation.

An image of the glint is also required, which can be a separate glint image taken at a different time or with a different camera. Alternatively the glint image can be one and the same as the pupil image i.e., despite the fact that the glint and pupil exposures are independently controlled, both an image of the glint and an image of the pupil are captured during one exposure as a superimposed or composite image.

The glint in the glint image may optionally be caused by a separate glint illuminator. For example, two or more pupil illuminators may be disposed to the sides of the lens to more evenly illuminate the face, with their glints being extinguished by the process of cross-polarization, so as to not obscure the image of the pupil with multiple glints. Then one or more glint illuminators are used to produce a controlled glint or glints. Alternatively, the glint (or glints) in the image may be caused by the pupil illuminator, except that its otherwise excessive brightness is attenuated by adjusting the camera polarizer to substantially but not complete extinguish the glint.

After capturing the image or images, software as practiced in the old art is used to find the centers of the pupil and glint. The distance between their centers is then used as an indicator of the person's direction of gaze. When the final intent is to determine a user's gaze point, as for example where he or she is looking on a computer monitor, the geometric relationship between the user's eyes and the screen is also taken into account, as again is practiced in the old art.

### BRIEF DESCRIPTION OF THE DRAWINGS

FIG. 1 is a perspective and partially exploded view of a user and a gaze-tracking system in accordance with the present invention.

FIG. 2 is a region of a glint and pupil image in accordance with the prior art and captured using a medium quality lens.

FIG. 3A is a region of a pupil image obtainable using one embodiment of the system of FIG. 1.

FIG. 3B is a region of a glint image obtainable using one embodiment of the system of FIG. 1.

FIG. 3C is a region of a glint and pupil image obtainable using one embodiment of the system of FIG. 1.

FIG. 4 is a schematic diagram of the gaze-tracking system of FIG. 1.

FIG. 5 is a flow chart of a gaze-tracking process in accordance with one exemplary embodiment of the invention and implemented in the system of FIG. 1.

FIG. 6 is a flow chart of a gaze tracking process in accordance with a different exemplary embodiment of the invention and implemented in the system of FIG. 1.

### DETAILED DESCRIPTION

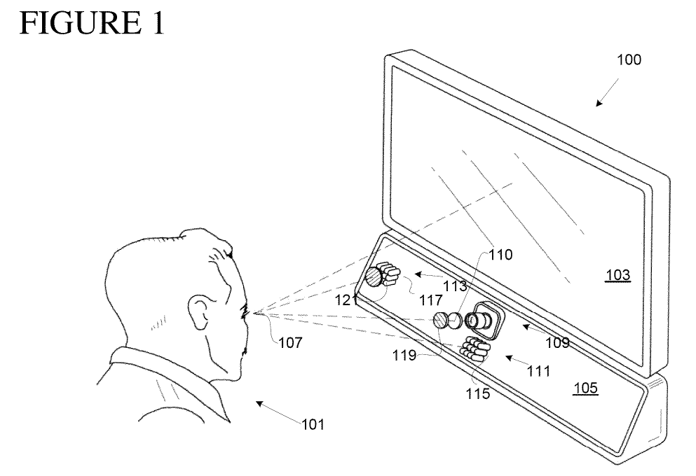

FIG. 1 is a view of a user 101 interacting with a computer system 100 according to one embodiment of the current invention. System 100 includes a display 103 and a gaze-tracking system 105 for tracking the motion of user eye 107. Gaze-tracking system 105 includes a camera 109, a “glint” illuminator 111, and a “pupil” illuminator 113. Illuminators 111 and 113 include respective LED arrays 115 and 117, which both emit infra-red light invisible to eye 107 but detectable by camera 109. Illuminator 113 is sufficiently bright that it can overcome ambient light.

Camera 109 includes a near infra-red (NIR) filter 110 to block visible light, diminishing interference in the camera images from ambient sources of light. LED arrays 115 and 117 illuminate the eye from below with NIR light. In alternative embodiments, visible light is used to illuminate, the illuminators are not below eye level, and exposures are varied in the camera.

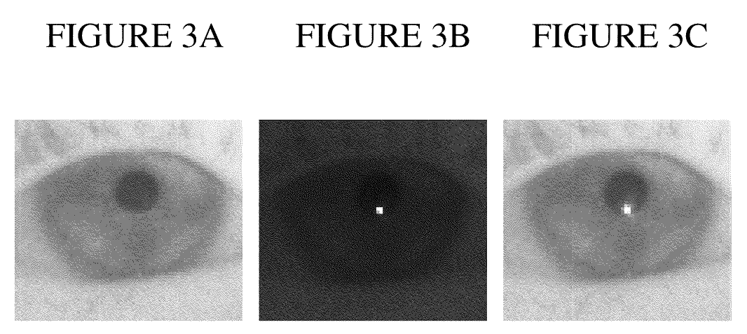

Camera 109 includes a polarizing filter 119 mounted thereon. Pupil illuminator 113 includes a polarizing filter 121 mounted thereon. In an alternative embodiment, the incoming polarizer is in front of the camera, but not mounted to it. Polarizing filters 119 and 121 are cross-polarized (opposite linear or opposite circular polarizations) so that reflections of light from array 113 off of eye 107 are attenuated relative to light scattered by eye 107. Since a glint is a reflection, while scattered light is used to image a pupil, the polarizers have the effect of removing glint from the pupil image, making it easier to determine pupil position precisely. This effect can be recognized by comparing the glint image region of FIG. 3B with the pupil image region of FIG. 3A. In the illustrated embodiment, polarizing filters 119 and 121 are linear polarizers. Alternative embodiments use polarizing beam splitters and circular polarizers.

Camera 109 images an approximately 10″ wide swath of the face, at a resolution of 1280 pixels. This means that the pixels are only 0.008″ apart, and a 0.10″ pupil will only be resolved by 13 pixels. Further the glint may only move approximately 0.1″ across the eye, or 13 pixels, as one looks from side to side on a 10″ wide screen viewed from 24″. Accordingly, the glint and pupil positions are measured with a precision of approximately 0.1 pixel. That allows a resolution of approximately 130 points across the screen, which, even in the presence of some jitter, is sufficient for most applications of gaze tracking such as cursor control.

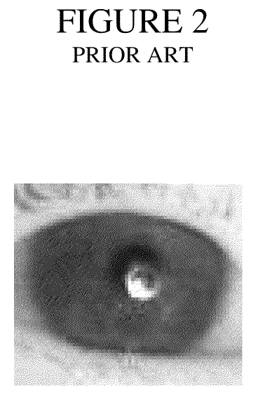

The advantage of obtaining separate glint and pupil images is to independently control their exposure. Were this not the case, the combined exposure of the glint and pupil might look more like that of the prior art as depicted in FIG. 2, captured using a $55 Edmund Optics #54-854 video imaging lens. In it, both the iris is rendered too dark and the glint too bright. While a more expensive lens will reduce the effect, nonetheless no exposure is possible that will allow the pupil and glint to each be optimally exposed as in FIG. 3A and FIG. 3B.

The pupil image of FIG. 3A can for example correspond to an exposure that is at least 50% greater than that of the glint image of FIG. 3B, or as much as ten or more times greater. If the glint and pupil images are separate images, then the pupil can be made to stand out against the iris in the pupil image, and the glint revealed without overexposure and flare in the glint image. Although a faint residue of the pupil and iris will persist in the glint image such as FIG. 3B, that will not detract from finding the exact centroid of the bright glint.

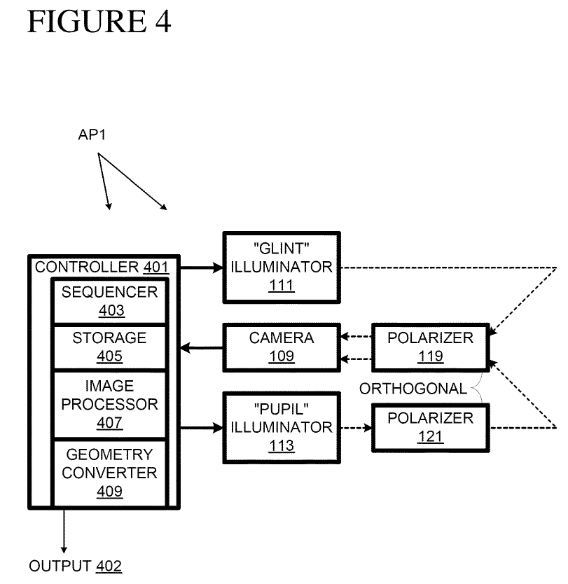

As shown in FIG. 4, gaze-tracking system AP1 includes a controller 401, camera 109, glint illuminator 111, pupil illuminator 113, and polarizers 119 and 121. Controller 401 includes a sequencer 403, storage media 405, an image processor 407, and a geometry converter 409. Storage media 405 are used for storing glint and pupil images, as well as for storing the results of image comparisons and analysis. Image processor 407 compares and analyzes glint and pupil images to determine glint and pupil centroids.

As in the prior art, the center of the pupil may be found by modeling it as a circle, and finding as many points on its periphery as possible to be able to determine its center with a high degree of accuracy. The problem faced by the prior art is the glint obscuring much of the lower periphery of the pupil, as depicted in FIG. 2. Therefore, often in the old art only the upper periphery of the pupil is relied upon for determining the vertical location of the pupil. This technique fails however if the upper periphery of the pupil becomes partially obscured by a drooping eyelid. The present invention addresses this problem by providing improved detail for the lower half of the pupil as seen in FIG. 3A and FIG. 3C, which is sufficient to make the vertical determination of the position of the pupil possible by relying solely on it.

Continuing with FIG. 4, geometry converter 409 converts these positions into a gaze point, yielding an output 402, e.g., a control signal such as a cursor control signal (as might otherwise be generated by a mouse or trackball).

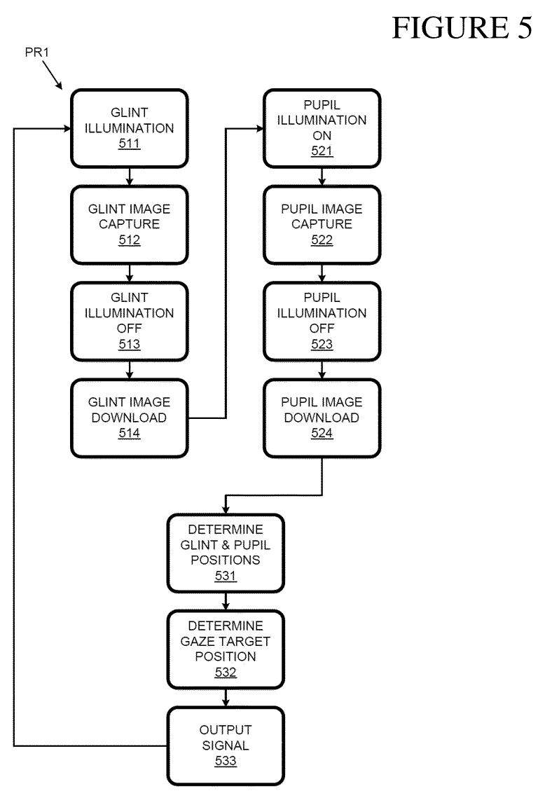

Sequencer 403 sequences process PR1, flow charted in FIG. 5, which is used to generate and analyze the glint and pupil images to determine gaze point. At process segment 511, sequencer 403 turns on glint illuminator 111 so as to illuminate eye 107. In practice, head movement must be allowed so illuminator 111 can be situated to illuminate an area much larger than one eye. While glint illuminator 111 is on, e.g., for a few tens of milliseconds, sequencer 403 commands camera 109 to capture an image at process segment 512. The result can be a glint image such as that shown in FIG. 3B. At process segment 513, glint illuminator 111 is turned off to save power and so as not to interfere with obtaining a pupil image. At process segment 514, the captured glint image is downloaded to storage media 405.

The brightness values in the glint image (and the pupil image) can range from zero to 255. In the glint image, the glint itself is typically at or near 255.

In process segments 521-524, sequencer 403 repeats segments 511-514 but instead to obtain a pupil image. At process segment 521, sequencer turns on pupil illuminator 113. The exposure is greater than that used for the glint image to obtain a brighter image despite the attenuating effects of the polarizers; for example, the pupil exposure can be at 200% to 2000% of the glint exposure, or even wider. This higher exposure more than compensates for the loss of light due to the effect of camera polarizer 121. The bright exposure for the pupil image also lifts the exposure level out of the noise floor of the camera and increases the detect ability of features such as the dividing line between a dark brown iris and a black pupil. In addition, the pupil illumination is polarized due to the presence of polarizing filter 121 to attenuate the glint, e.g., by three or four orders of magnitude.

At process segment 522, sequencer 403 commands camera 109 to capture an image, in this case a pupil image such as that represented in FIG. 3A. Any glint reflections are attenuated due to the cooperative action of polarizing filters 119 and 121, thus enhancing the detectability of the pupil. At process segment 523, pupil illumination is turned off. At process segment 524, the pupil image is downloaded to storage media 405. In alternative embodiments, the order of the process segments can be varied; for example, illuminators can be turned off after or during a download rather than before the downloading begins.

At process segment 531, the glint and pupil images are analyzed to determine glint and pupil positions. For example, centroids for the glint in the current glint image for the pupil in the current pupil image are obtained. The glint and pupil positions can be compared (subtracted) to determine a gaze point, or the same thing, a gaze target position, at process segment 532. In effect, the images are superimposed and treated as a single image so that the position of the pupil is determined relative to the position of the glint as in the prior art.

The process for finding the glint starts with searching for the brightest pixels. To eliminate bright pixels from glints off of glasses frames, a check can be made for a proximal pupil. Next, a correlation is performed on the glint by taking an expected image of the glint and translating it around for a best fit.

The pupil location can be determined and expressed in a number of ways. For example, the location of the pupil can be expressed in terms of the position of its center. The center can be determined, for example, by locating the boundary between the pupil and the iris and then determining the center of that boundary. In an alternative embodiment, the outer diameter of the iris (the boundary between the iris and the sclera) is used to determine the pupil position.

To compensate for movement between the times the glint and pupil images can be obtained, one or both of the glint and pupil positions can be extrapolated so that the two positions correspond to the same instant in time. To this end, one or more previously obtained glint and/or pupil images can be used. In an example, the cycle time for process PR1 is 40 ms and the pupil image is captured 10 ms after the corresponding glint image. Comparison of the glint positions indicates a head velocity of 4 pixels in 40 ms. This indicates a movement of 1 pixel in 10 ms. Thus, at the time the pupil image is captured, the glint position should be one pixel further in the direction of movement than it is in the actual current glint image. This extrapolated glint position is compared to the unextrapolated pupil position obtained from the pupil image.

At process segment 532, the calculations involved in determining a gaze target position or gaze point take into account the distance of the subject from the camera. This can be determined conventionally, e.g., using two cameras or measuring changes in the distance between the eyes. In other cases, an additional LED array can be used to make a second glint; in that case the distance between the glints can be measured.

A number of factors are taken into account to determine, from the glint and pupil positions in their respective images, where (e.g., on a computer screen) a person is actually gazing. These factors include the starting position of the user's eye relative to the screen and the camera, the instantaneous position of the user's eye with respect to the same, the curvature of the cornea, the aberrations of the camera lens, and the geometry of the screen. These mathematical corrections are performed in software, and are well known in the art. Often several corrections can be lumped together and accommodated by having the user first “calibrate” the system. This involves having the software position a target on several predetermined points on the screen, and then for each, recording where the user is gazing. Jitter is often removed by averaging or otherwise filtering several gaze points before presenting them.

At process segment 533, the determined gaze point can be used to generate output signal 402, e.g., a virtual mouse command, which can be used to control a cursor or for other purposes. Sequencer 403 then iterates process PR1, returning to process segment 511. Note that if the objective is a control signal rather than the gaze direction itself that is of interest, the gaze point need not be explicitly determined. It also may not be necessary to determine gaze target explicitly in an application that involves tracking head motion or determining the direction of eye movement. For example, in some applications, the direction of eye movement can represent a response (right=yes, left=no) or command.

In a different exemplary embodiment the glint and pupil are exposed in the same image, resulting in the image shown in FIG. 3C, which can be compared to the prior art depicted in FIG. 2. The image of FIG. 3C can be thought of as the superposition of the images 3A and 3B, as captured in the previously described embodiment. Ignoring nonlinearities, the imaging chip in the camera sums the pupil and glint exposures, pixel by pixel. The two image components, that of the pupil and iris, and that of the glint, are, like in the previously described embodiment, independently controlled such as to produce much the same benefit, a clearer image that better reveals the details of both the pupil and the glint.

The apparatus is the same one as shown in FIG. 1. The description of that figure pertains to this embodiment as well, with the understanding that the glint image and the pupil image are captured during the same exposure. The block diagram of FIG. 3 applies as well to this embodiment, with the change that the sequencer 403 captures both the pupil and the glint image concurrently.

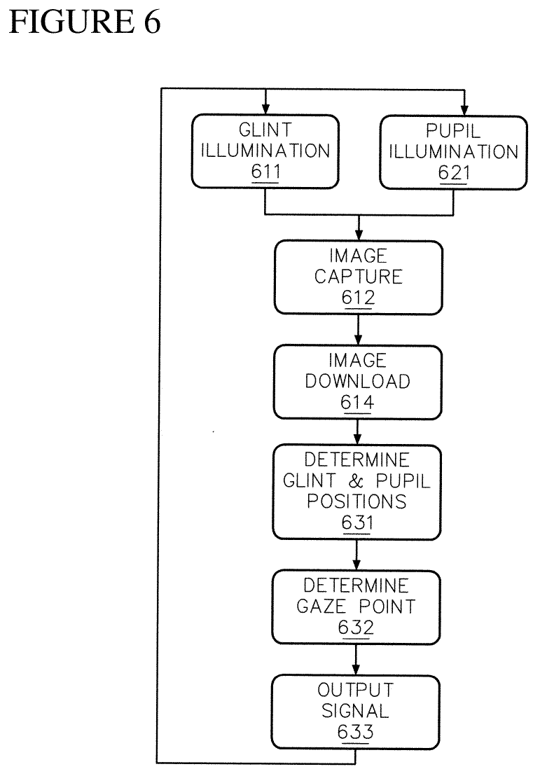

A flowchart for process of this embodiment is shown in FIG. 6. In it, process block 612 captures an image, using for example an exposure lasting 2 to 200 milliseconds. During the exposure interval, process block 611 turns on the glint illuminator 111. Since less light is required to expose the glint than the pupil and iris, the glint illuminator 111 may be a smaller source (fewer LEDs 115, for example), or it may not be turned on for the full duration of the capture.

Also during the capture interval, process block 621 turns on the pupil illuminator 113. In the interests of keeping the cycle time as short as possible, the pupil illuminator 113 can be left on for the full duration of the exposure, and the exposure time set only long enough to achieve the desired exposure.

In process block 614 the captured image is downloaded to storage block 405 in the controller 401. If the camera 109 is able to download an old image concurrently with capturing a new one, then the downloading time beneficially does not lengthen the cycle time.

In process block 631, software is used as in the prior art to determine the centroid of the glint and the center of the pupil. Locating the centroid of the glint is straightforward because the glint is the brightest object in the image. As is known in the art, a template image of the expected distribution of the glint can be 2-dimensionally correlated with the glint in the image to find the point of best alignment. The template can for example be 7×7 pixels in size. The correlation values for each offset of the template vs. the image are recorded. Since the best fit will usually lie between integer values of offsets between the template and the glint image, correlation values for different offsets are interpolated between to determine the fractional pixel offset representative of the best horizontal and vertical fit.

Locating the center of the pupil is slightly more complicated because at times the glint can variously intersect or lie inside the pupil in the image. Nonetheless the center of the pupil is located the same as has been practiced in the old art, by modeling the pupil as a circle, and determining the point where the circle best aligns with the pupil's periphery. The difference lies in that in the present invention, the glint has been made much smaller in the image, and rendered without lens flare or other artifacts, leaving a much cleaner image of the pupil to analyze. As with the glint determination, the location of the center of the pupil is determined to a fraction of one pixel.

In a different exemplary embodiment, polarized light is used to allow more even illumination of the face. For example, multiple pupil illuminators 113 may be disposed to both sides of the camera 109, to better illuminate the format of the camera 109 which may be wider horizontally than vertically. These illuminators would normally produce undesirable multiple glints, causing the periphery of the pupil at times to become even more obscured. This is prevented in this embodiment however by making the orientations the same for the polarizers 121 on these pupil illuminators 113, and setting the orientation of the camera polarizer 119 at an angle of 90 degrees to the pupil polarizers 121, to attenuate these glints. A glint illuminator 111, which of course does not have a polarizer, is used to produce a glint in an image.

In a different exemplary embodiment, a separate glint illuminator is not required. Instead the pupil illuminator 113 illuminates both the pupil and the glint, however not in the mode practiced in the old art. Instead the camera polarizer 119 is set to nearly but not completely eliminate the glint. By judiciously setting the polarizer 119, the glint image and the pupil image can each be exposed in a different and controlled manner. The glint exposure is made much less than the pupil exposure, and the identical image of FIG. 2D is produced, the same as with a previously described embodiment.

The invention provides for many variations upon and modifications to the embodiments described above. For example, the processes of FIG. 5 and FIG. 6 may be preceded by a search for the region of the eyes. This mode is useful when first acquiring the eyes, or reacquiring them after a user blinks. This mode may use different lighting conditions, such as overly bright controlled lighting from a glint illuminator 111, for the purpose of overwhelming ambient light and roughly locating the eyes. In one embodiment, the pupil illuminator includes more than one array of LEDs, e.g., more than one pupil illuminator is used. In another embodiment, more than one glint illuminator is used, and may be used to determine the distance from the user to the camera. In another embodiment, the pupil illuminator and/or glint illuminator includes a circular array of LEDs around the camera lens. For example, the pupil illuminator can include a circular array around the lens and an array of LEDs away from the lens. The circular array can be used when a “red pupil” (AKA “bright pupil”) mode is selected, while the remote array can be used when “black pupil” (AKA “dark pupil”) mode is selected. Also, various arrangements (positions and angles) of illuminators can be used to minimize shadows (e.g., by providing more diffuse lighting) and to reduce the effect of head position on illumination. Illuminators can be spread horizontally to correspond to a landscape orientation of the camera. Depending on the embodiment, the camera can be helmet mounted or “remote”, i.e., not attached to the user.

To reduce or eliminate the need for motion compensation when separate glint and pupil images are used, the latency between the images can be minimized. In an alternative embodiment, the camera permits two images to be captured without downloading in between. In another embodiment, glint and pupil images are captured by separate cameras to minimize the delay. For example, two cameras may be rendered coaxial through the use of a beamsplitter. In some embodiments, polarization is achieved using polarizing beam splitters.

Any reference to a “pupil image” or a “glint image” is meant to include images showing more extensive portions of the eye, or the face, or of the environment. Further, any reference to finding the center of the pupil is meant to include doing so by finding the center of either or both of the inner and outer peripheries of the iris. Since the outer periphery of the pupil is the same as the inner periphery of the iris, terms such as “pupil image” and “pupil and iris image” are meant to be interchange.

Drooping eyelids can be accommodated in all of the embodiments. For example, during setup, a system might be set rely on the lower periphery of the pupil in determining the vertical center of the pupil. On the other had if droop is not anticipated, then the vertical center can be calculated using either the upper periphery, or both the upper and the lower peripheries.

In any of the above embodiments, a preliminary image can be captured under relaxed conditions for the sole purpose only of localizing the eyes and the corneal glint in the image of the user's face. The preliminary image may also optionally be used to determine the distance between the user and the camera. Subsequent images then only need to be analyzed over a smaller region of interest that includes one or both eyes.

In this specification, related art is discussed for expository purposes. Related art labeled “prior art” is admitted prior art; related art not labeled “prior art” is not admitted prior art. The embodiments described above, variations thereupon, and modifications thereto are within the subject matter defined by the following claims.

1. A process comprising:
illuminating one eye to produce a glint on said eye;
obtaining a glint image of said eye showing said glint on said eye;
further illuminating said eye using polarized light;
obtaining, through a polarizer that can attenuate reflected polarized light, a pupil image of said eye;
determining one glint position at least in part from said glint image, and
determining one pupil position at least in part from said pupil image.

2. A process as recited in claim 1 wherein said glint image and said pupil image are the same image.

3. A process as recited in claim 1 wherein said glint image and said pupil image are different images.

4. A process as recited in claim 1, additionally performing, as a preceding step, illuminating said one eye to produce a first glint on said eye; obtaining a preliminary image of said first glint; and analyzing said preliminary image so as to approximately locate said first glint within said preliminary image.

5. A process as recited in claim 1 further comprising determining a gaze point of said eye at least in part by comparing said glint position with said pupil position.

6. A process as recited in claim 1 further comprising determining a gaze direction of said eye at least in part by comparing said glint position with said pupil position.

7. A process as recited in claim 1 wherein the exposure with which said pupil image is obtained is different than the exposure with which said glint image is obtained.

8. A process as recited in claim 1 wherein said further illuminating said eye using polarized light involves multiple illuminators, wherein each of said multiple illuminators emits light of the same polarization, and wherein a first one of said multiple illuminators is disposed at one side of said polarizer and a second one of said multiple illuminators is disposed at the opposite side of said polarizer.

9. A process as recited in claim 3 wherein at least one of said pupil position and said glint position is an extrapolated position.

10. A process as recited in claim 9 additionally performing, as a preceding step, either illuminating said at least one eye to produce a first glint on said eye using polarized light and obtaining a corresponding glint image, or illuminating said at least one eye with polarized light and obtaining through a polarizer a corresponding pupil image, wherein either said corresponding glint image or said corresponding pupil image is used in obtaining said extrapolated position.

11. A system comprising:
a camera for obtaining a glint image and a pupil image;
a glint illuminator for illuminating an eye to produce a glint that is represented in said glint image;
a pupil illuminator for illuminating said eye so that a pupil is represented in said pupil image;
polarizers in an optical path between said pupil illuminator and said camera, said polarizers cooperating to attenuate light reflected by said eye relative to light scattered by said eye; and
a controller which causes said glint and pupil images to be obtained within a total time interval of 0.2 s, and analyzes said images so as to compare at least one glint position with at least one pupil position, said at least one glint position being determined at least in part from said glint image, said at least one pupil position being determined from said pupil image.

12. A system as recited in claim 11 wherein said glint image and said pupil image are the same image.

13. A system as recited in claim 11 wherein said glint image and said pupil image are different images.

14. A system as recited in claim 11 wherein said controller determines a gaze point at least in part as a function of said glint and pupil images.

15. A system as recited in claim 11 wherein said controller controls the exposures for said glint and pupil images so that the overall brightness of said pupil image is at least twice that of the overall brightness of said glint image.

16. A system as recited in claim 11 wherein at least one of said polarizers is a polarizing beam splitter.

17. A system as recited in claim 11 wherein said polarizers are linear polarizers.

18. A system as recited in claim 11 wherein said polarizers are circular polarizers.

19. A system as recited in claim 11 wherein said illuminators provide infrared light.

20. A system as recited in claim 11 wherein said controller extrapolates at least one of said glint and pupil positions to obtain modified glint and pupil positions corresponding to the same instant in time.
